(19) United States
(12) Patent Application Publication
(10) Pub. No.: US 2021/0315230 A1
(43) Pub. Date: Oct. 14, 2021

(54) PRODUCT AND METHOD FOR MAKING DISSOLVING CHEWING GUM

(71) Applicant: Camille Varlet, New York, NY (US)

(72) Inventor: Camille Varlet, New York, NY (US)

(21) Appl. No.: 17/230,113

(22) Filed: Apr. 14, 2021

Related U.S. Application Data
(60) Provisional application No. 63/009,539, filed on Apr. 14, 2020.
(60) Provisional application No. 63/199,407, filed on Dec. 23, 2020.

(51) Int. Cl. A23G 4/02; A23G 4/10; A23G 4/06; A23G 4/20; A61K 9/68

(52) U.S. Cl. CPC A23G 4/02; A23G 4/10; A23G 4/068; A23V 2002/00; A23G 4/066; A61K 9/0058; A23G 4/20

(57) ABSTRACT

A new dissolving chewing gum product and methods to make the new chewing gum are described. A first method includes: creating a base for the dissolving chewing gum by heating, cooking, and mixing a liquid, a chew/texture component, and a texture maintenance component; adding additional components to the base; cooling the base to incorporate sensitive components; and forming individual pieces from the base. A second method occurs without a cooking step and includes: creating a base for the dissolving chewing gum by mixing a chew/texture component/texture maintenance component, adding additional components to the base, forming individual pieces of the dissolving chewing gum from the base, covering the individual pieces of the dissolving chewing gum with a coating, and adding a polish and/or a wax to the individual pieces of the dissolving chewing gum.

### CROSS-REFERENCE TO RELATED APPLICATIONS SECTION

This application is a U.S. Non-Provisional patent application that claims priority to U.S. Provisional Patent Application Ser. 63/009,539 filed on Apr. 14, 2020 and U.S. Provisional Patent Application Ser. 63/199,407 filed on Dec. 23, 2020, the entire contents of which are hereby incorporated by reference in their entirety.

### FIELD OF THE EMBODIMENTS

The field of the embodiments relate to a new type of and the methods to make this new type of chewing gum. In particular, the instant invention provides a chewing gum that dissolves as the user chews the chewing gum until it is completely gone, and is plant-based, plastic-free and sugar-free. This chewing gum can also contain and be used to deliver active ingredients, which are released as the user chews the gum, for various functionalities and benefits.

### BACKGROUND OF THE EMBODIMENTS

Many gums today contain sugar or artificial sweeteners and other artificial ingredients. Some can also contain animal product such as gelatin, shellac, or other materials. Further, some gum bases also contain plastic. In addition, gum is considered one of the top sources of litter in the streets, due to the lack of dissolvability of the formula. At times, users can find themselves with no place to spit out their gum, which is inconvenient. Thus, what is needed is a product that addressed all of these issues. Provided herein is a plant-based and zero-sugar gum that dissolves as the user chews the gum. Moreover, the gum releases active ingredients as the user chews the gum, which may provide numerous functionalities to the user, such as: removing bad breath molecules of the user, providing energy to the user, reducing anxiety of the user, providing vitamins or probiotics to the user, etc.

### SUMMARY OF THE EMBODIMENTS

This summary is provided to introduce a selection of concepts in a simplified form that are further described below in the Detailed Description section. This summary is not intended to identify key features or essential features of the claimed subject matter, nor is it intended to be used as an aid in determining the scope of the claimed subject matter.

A first embodiment of the present invention describes a dissolving chewing gum and a method to make the dissolving chewing gum. The method includes numerous process steps, such as: creating a base for the dissolving chewing gum by heating, cooking, and mixing a liquid, a chew/texture component, and a texture maintenance component. The liquid comprises water. The chew/texture component comprises a fat, a thickening agent, and/or a gelling agent, among other chew/texture components not explicitly described herein. The texture maintenance component may be: a canola lecithin, a soy lecithin, or a sunflower lecithin, among other texture maintenance components not explicitly described herein.

The method then includes adding additional components to the base. The additional components may include a sweetening/texture component. The sweetening/texture component may be: a crystalline maltitol, a polyglycitol syrup, a polyol syrup, or a corn syrup, among other sweetening/texture components not explicitly described herein, and additionally may include or be comprised of sugar alcohols that may include: sorbitol, mannitol, maltitol, lactitol, erythritol, or a sugar substitute, among other sugar alcohols not explicitly described herein. The sugar substitute may include stevia, xylitol, and/or isomalt, or a combination of any of these, among other sugar substitutes not explicitly described herein.

Next, the method includes: cooling the base to incorporate a sensitive component. The sensitive component is preferably not added sooner because it would become compromised or reduced in its benefit to the formula. The sensitive component may include a natural flavoring component and/or an active component. The natural flavoring component may include a peppermint flavoring component, a spearmint flavoring component, an orange flavoring component, a strawberry flavoring component, a cinnamon flavoring component, a watermelon flavoring component, a bubble gum flavoring component, a ginger flavoring component, a lemon flavoring component, a licorice flavoring component, a lemongrass flavoring component, a coffee flavoring component, or a grapefruit flavoring component, among other flavoring components not explicitly described herein. The active component includes: caffeine, CBD, a botanical component, collagen, pea protein, a pre-biotic, a pro-biotic, an enzyme, calcium carbonate, magnesium hydroxide, acetaminophen, zinc, vitamin C, B vitamins, Vitamin A, magnesium, xylitol, and/or loratadine, among other active components not explicitly described herein.

The method then includes forming individual pieces of the dissolving chewing gum from the base. In some examples, the method optionally includes an additional step of pre-sealing each of the individual pieces of the dissolving chewing gum. The method may additionally include: adding an engrossing syrup to each of the individual pieces of the dissolving chewing gum, adding a finishing syrup and/or a coloring component to each of the individual pieces of the dissolving chewing gum, and/or adding a coating component, a polish, and/or a wax to each of the individual pieces of the dissolving chewing gum.

A second embodiment of the present invention describes the dissolving chewing gum and a method to make the dissolving chewing gum that occurs without a cooking step or process. The method includes creating a base for the dissolving chewing gum by mixing a chew/texture component, and a texture maintenance component. The texture maintenance component may include a canola lecithin, a soy lecithin, and/or a sunflower lecithin, among other components. The chew/texture component may include a fat, and/or a thickening agent and/or a gelling agent. In examples, the fat is a stearine or a palm oil. In some examples, the thickening and gelling agents are starch, some examples being corn starch and modified food starch, among other components.

The method may additionally include adding additional components to the base. The additional components may include: a sweetening/texture component, another texture maintenance component, a natural flavoring component, and/or an active component. The sweetening/texture component may include: a crystalline maltitol, a polyglycitol syrup, a polyol syrup, or a corn syrup, among other sweetening/texture components not explicitly listed herein. The polyol syrup may include a sugar alcohol, among other polyol syrups not explicitly listed herein and additionally may include or be comprised of sugar alcohols that may include: sorbitol, mannitol, maltitol, lactitol, erythritol, or a sugar substitute, among other sugar alcohols not explicitly listed herein. The sugar substitute may include stevia, xylitol, and/or isomalt, or a combination of any of these, among other sugar substitutes not explicitly listed herein.

Further, the natural flavoring component may include: a peppermint flavoring component, a spearmint flavoring component, an orange flavoring component, a strawberry flavoring component, a cinnamon flavoring component, a watermelon flavoring component, a bubble gum flavoring component, a ginger flavoring component, a lemon flavoring component, a licorice flavoring component, a lemongrass flavoring component, a coffee flavoring component, or a grapefruit flavoring component, among other flavoring components not explicitly listed herein.

The active component may include: caffeine, CBD, a botanical component, collagen, pea protein, a pre-biotic, a probiotic, an enzyme, calcium carbonate, magnesium hydroxide, acetaminophen, zinc, vitamin C, B vitamins, Vitamin A, magnesium, antioxidant, xylitol, and/or loratadine, among other active components not explicitly listed herein. The active component is released as a user chews the dissolving chewing gum product to provide a benefit to the user. The benefit to the user may include: removing bad breath molecules of the user, providing energy to the user, reducing anxiety of the user, delivering vitamins to the user, delivering protein to the user, delivering health and/or wellness benefits the user, delivering plaque or biofilm reduction or removal benefits to the user, and/or providing medical components to the user, among other benefits not explicitly listed herein.

The method also includes forming individual pieces of the dissolving chewing gum from the base, covering the individual pieces of the dissolving chewing gum with a coating, and adding a polish and/or a wax to the individual pieces of the dissolving chewing gum.

In general, the present invention succeeds in conferring the following benefits and objectives.

It is an object of the present invention to provide a chewing gum that dissolves and can deliver flavor, as well as active ingredients, during the chewing process.

It is an object of the present invention to provide a chewing gum that chews for at least 10 seconds in the mouth.

It is an object of the present invention to provide a chewing gum that is made without sugar.

It is an object of the present invention to provide a chewing gum that is made without artificial sweeteners or other artificial ingredients.

It is an object of the present invention to provide a chewing gum that contains no plastic and no animal product/is plant based.

### BRIEF DESCRIPTION OF THE DRAWINGS

FIG. 1 depicts a block diagram of a method to make a dissolving chewing gum product that includes a cooking step, according to at least some embodiments described herein.

FIG. 2 depicts a block diagram of a method to make a dissolving chewing gum product that does not include a cooking step, according to at least some embodiments described herein.

### DETAILED DESCRIPTION OF THE PREFERRED EMBODIMENT

Various embodiments are described more fully below with reference to the accompanying drawing, which forms a part hereof, and which shows specific exemplary embodiments. However, embodiments may be implemented in many different forms and should not be construed as limited to the embodiments set forth herein; rather, these embodiments are provided so that this disclosure will be thorough and complete, and will fully convey the scope of the embodiments to those skilled in the art. The following detailed description is, therefore, not to be taken in a limiting sense.

Reference in the specification to “one embodiment” or to “an embodiment” means that a particular feature, structure, or characteristic described in connection with the embodiments is included in at least one embodiment of the invention. The appearances of the phrase “in one embodiment” in various places in the specification are not necessarily all referring to the same embodiment.

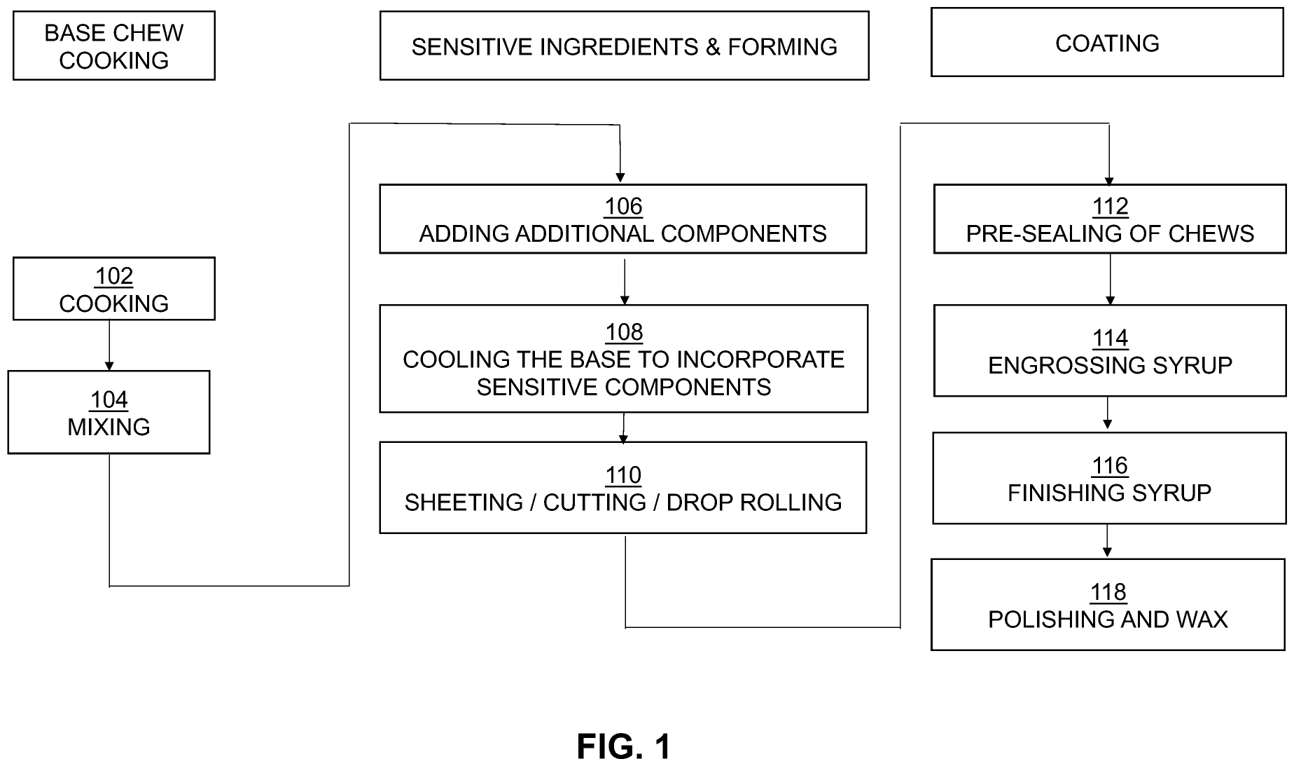

The method of FIG. 1 includes numerous process steps. It should be appreciated that the process steps described herein may be performed in a differing order then what is described. For example, two process steps in succession may, in fact, be executed substantially concurrently, or the process steps may sometimes be executed in the reverse order, depending upon the process step involved. Moreover, it should be appreciated that the listing of the materials herein is non-exhaustive and other materials, not explicitly listed herein, are contemplated by Applicant.

As shown in FIG. 1, three processes are included in the method to make the dissolving chewing gum. The three processes include: (1) a base product cooking process/a process of creating a chewy base, (2) a process of incorporating sensitive ingredients, and (3) a coating process, such as a hard panning process. It should be appreciated that the process steps of FIG. 1 described herein may be performed in a differing order then what is described. For example, two process steps in succession may, in fact, be executed substantially concurrently depending upon the process step involved. Moreover, it should be appreciated that the listing of the materials herein is non-exhaustive and other materials, not explicitly listed herein, are contemplated by Applicant.

The process step 102 includes cooking/heating materials for a base for the dissolving chewing gum product. The materials for the base may include: a liquid, a chew/texture component, and a texture maintenance component. The liquid may be a purified or a non-purified liquid. In examples, the liquid may be water. The water may be used for component incorporation and/or solubility, but it is not necessary.

The texture maintenance component may include a canola lecithin, a soy lecithin, and/or a sunflower lecithin, among other components. The canola lecithin may help to hold fat in the chewing gum and prevent an oily texture of the chewing gum. The chew/texture component may include a fat, a thickening agent, and/or a gelling agent. In examples, the fat is a stearine or a palm oil. In some examples, the gelling agent is an agar/locust bean gum.

The materials may additionally or optionally comprise a sweetening/texture component, among other components. The sweetening/texture component may include a crystalline maltitol, a polyglycitol syrup, and/or a polyol syrup, among other components. In some examples, the sweetening/texture component may include a corn syrup. The polyol syrup may include a sugar alcohol, among other polyol syrups not explicitly listed herein and additionally may include or be comprised of sugar alcohols that may include: sorbitol, mannitol, maltitol, lactitol, erythritol, or a sugar substitute, among other sugar alcohols not explicitly listed herein. The sugar substitute may include stevia, xylitol, and/or isomalt, or a combination of any of these, among other sugar substitutes not explicitly listed herein. In some examples, utilizing the polyglycitol or the polyol syrup with the crystalline maltitol balances crystallization and provides a favorable end texture of the dissolving chewing gum product.

In some examples, the base product is heated at a temperature until a target weight loss is achieved. The temperature is in a range of approximately 120° C. to approximately 250° C. Optimal moisture level is 5% or above, as the optimal moisture level being below 5% results in a chewing gum product that is too firm for a user to chew.

The process step 102 is followed by a process step 104 that includes mixing the materials for the base for the dissolving chewing gum product. The materials may be mixed at a temperature in a range of approximately 100° C. to approximately 170° C.

The process step 104 is followed by a process step 106 that includes adding additional components to the base product. The additional components may target chew/texture. If incorporating one or more sensitive components, the process step 106 is followed by a process step 108 that includes cooling the base product to incorporate the one or more sensitive components. It should be appreciated that the process step 106 and the process step 108 are part of the mixing process described herein. The sensitive components include a flavoring component and optionally active components, among others.

The flavoring component may include a natural flavoring component. In examples, the natural flavoring component may include: a peppermint flavoring component, a spearmint flavoring component, an orange flavoring component, a strawberry flavoring component, a cinnamon flavoring component, a watermelon flavoring component, a bubble gum flavoring component, a ginger flavoring component, a lemon flavoring component, a licorice flavoring component, a lemongrass flavoring component, a coffee flavoring component, and/or a grapefruit flavoring component, and/or a combination of flavoring components, among others. The active component is released as the user chews the dissolving chewing gum product to provide various functionalities and benefits to the user. In examples, the active component may include caffeine, CBD, a botanical component, collagen, pea protein, a pre-biotic, a pro-biotic, an enzyme, calcium carbonate, magnesium hydroxide, acetaminophen, zinc, vitamin C, B vitamins, Vitamin A, magnesium, xylitol, and/or loratadine, among others. In additional examples, the active component is configured to remove bad breath molecules of a user chewing the dissolving chewing gum product, provide energy to the user chewing the dissolving chewing gum product, reduce anxiety of the user chewing the dissolving chewing gum product, deliver vitamins to the user chewing the dissolving chewing gum product, deliver protein to the user chewing the dissolving chewing gum product, deliver health and/or wellness benefits to the user chewing the dissolving chewing gum product, deliver plaque or biofilm reduction or removal benefits to the user chewing the dissolving chewing gum product and/or provide medical components to the user chewing the dissolving chewing gum product.

The process step 108 may be followed by a process step 110 that includes forming individual pieces of the dissolving chewing gum from the base. In examples, this may include sheeting, cutting, or drop rolling the base. A process step 112, a process step 114, a process step 116, and a process step 118 are optional and may follow the process step 110.

For example, the process step 112 may include optionally pre-sealing each of the individual pieces of the dissolving chewing gum. The process step 114 may include adding an engrossing syrup to each of the individual pieces of the dissolving chewing gum. The process step 116 may include adding a finishing syrup and/or a coloring component to each of the individual pieces of the dissolving chewing gum. The process step 118 may include applying a coating component, the coloring component (e.g., for hard coating/panning), a polish, and/or a wax to each of the individual pieces of the dissolving chewing gum. The coloring component may include sucrose esters and/or a titanium dioxide, among other components. The sucrose esters may be useful in promoting finer crystal sizes, which appear white in color. The coating component may include a crystalline sorbitol, a sugar, a maltitol, an isomalt, and/or another polyol, among other components.

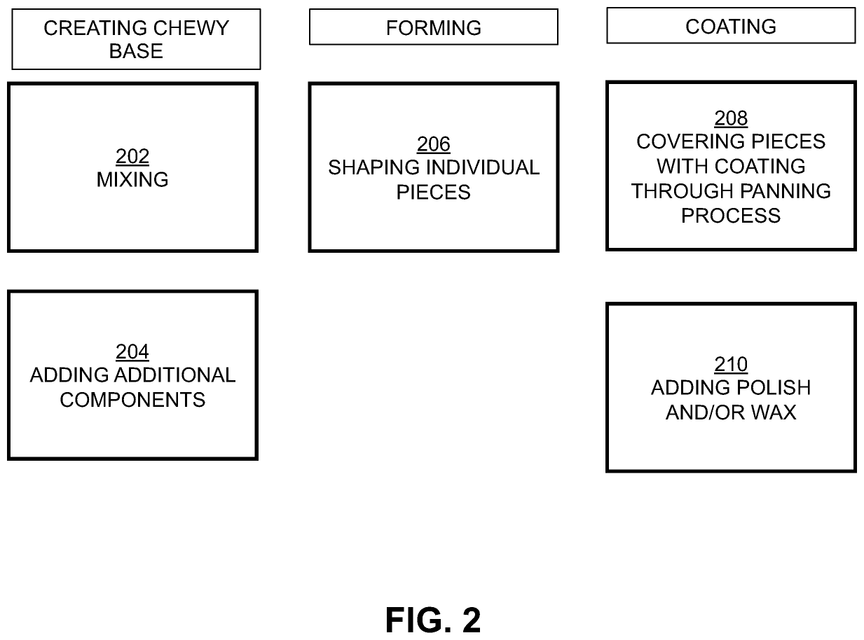

The method of FIG. 2 is substantially similar to the method of FIG. 1. However, the method of FIG. 2 does not include any cooking processes. It should be appreciated that the process steps described herein for the method of FIG. 2 may be performed in a differing order then what is described. For example, two process steps in succession may, in fact, be executed substantially concurrently, or the process steps may sometimes be executed in the reverse order, depending upon the process step involved. Moreover, it should be appreciated that the listing of the materials herein is non-exhaustive and other materials, not explicitly listed herein, are contemplated by Applicant.

The method of FIG. 2 includes three processes: a process of creating a chewy base, a forming process, and a coating process. The process of creating a chewy base includes a process step 202 and a process step 204. The forming process includes a process step 206. The coating process includes a process step 208 and a process step 210.

The process step 202 includes mixing materials for a chewy base for the dissolving chewing gum product. The materials for the base may include: a chew/texture component and a texture maintenance component.

The texture maintenance component may include a canola lecithin, a soy lecithin, and/or a sunflower lecithin, among other components not explicitly listed herein. The canola lecithin may help to hold fat in the chewing gum and prevent an oily texture of the chewing gum. The chew/texture component may include a fat, a thickening agent, and/or a gelling agent, among other components not explicitly listed herein. In examples, the fat is a stearine or a palm oil. In some examples, the thickening and gelling agents are starch, some examples being corn starch and modified food starch, among other components.

The process step 202 is followed by a process step 204 that includes adding additional components to the base product. The additional components may comprise a sweetening/texture component, another texture maintenance component, a natural flavoring component, and/or an active component. The sweetening/texture component may include a crystalline maltitol, a polyglycitol syrup, and/or a polyol syrup, among other components not explicitly listed herein. In some examples, the sweetening/texture component may include a corn syrup. The polyol syrup may include a sugar alcohol, among other polyol syrups not explicitly listed herein and additionally may include or be comprised of sugar alcohols that may include: sorbitol, mannitol, maltitol, lactitol, erythritol, or a sugar substitute, among other sugar alcohols not explicitly listed herein. The sugar substitute may include stevia, xylitol, and/or isomalt, or a combination of any of these, among other sugar substitutes not explicitly listed herein. In some examples, utilizing the polyglycitol or the polyol syrup with the crystalline maltitol balances crystallization and provides a favorable end texture of the dissolving chewing gum product.

The flavoring component may include a natural flavoring component. In examples, the natural flavoring component may include: a peppermint flavoring component, a spearmint flavoring component, an orange flavoring component, a strawberry flavoring component, a cinnamon flavoring component, a watermelon flavoring component, a bubble gum flavoring component, a ginger flavoring component, a lemon flavoring component, a licorice flavoring component, a lemongrass flavoring component, a coffee flavoring component, and/or a grapefruit flavoring component, and/or a combination of flavoring components, among others.

In examples, the active component may include caffeine, CBD, a botanical component, collagen, pea protein, a pre-biotic, a pro-biotic, an enzyme, calcium carbonate, magnesium hydroxide, acetaminophen, zinc, vitamin C, B vitamins, Vitamin A, magnesium, xylitol, and/or loratadine, among others. The active component is released as the user chews the dissolving chewing gum product to provide various functionalities and benefits to the user, such as: removing bad breath molecules of the user chewing the dissolving chewing gum product, providing energy to the user chewing the dissolving chewing gum product, reducing anxiety of the user chewing the dissolving chewing gum product, delivering vitamins to the user chewing the dissolving chewing gum product, delivering protein to the user chewing the dissolving chewing gum product, delivering health and/or wellness benefits to the user chewing the dissolving chewing gum product, delivering plaque or biofilm reduction or removal benefits to the user chewing the dissolving chewing gum product and/or providing medical components to the user chewing the dissolving chewing gum product.

The process step 204 may be followed by a process step 206 that includes forming individual pieces of the dissolving chewing gum from the chewy base. In examples, this may include sheeting, cutting, or drop rolling the base. A process step 208 and a process step 210 may follow the process step 206.

An optional process step may occur that may include pre-sealing each of the individual pieces of the dissolving chewing gum. The process step 208 may include covering the individual pieces of the dissolving chewing gum with a coating through a panning process. The process step 210 may include adding a coloring component, a polish, and/or a wax to the individual pieces of the dissolving chewing gum. The coloring component may include sucrose esters and/or a titanium dioxide, among others not explicitly listed herein. The sucrose esters may be useful in promoting finer crystal sizes, which appear white in color. The coating component may include a crystalline sorbitol, a sugar, a maltitol, an isomalt, and/or another polyol, among other components not explicitly listed herein.

The chewing gum produced by the method of FIG. 1 and the method of FIG. 2 provides numerous benefits as compared to other gums currently on the market. For example, the chewing gum dissolves quickly as the user chews the chewing gum. Moreover, chewing gum encourages saliva production and, in itself, has oral care benefits to the user chewing the gum. The chewing gum is convenient, sugar-free, and without any plastic, without artificial sweeteners or other artificial ingredients, and as such, is a healthy formula and better for the environment as compared to typical chewing gum products on the market. It can additionally deliver functionality and active ingredients in a easy to ship, easy to carry and discrete to consume format. Moreover, since this chewing gum is dissolving, it is eco-friendly and will not create litter on the streets.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A method to make a dissolving chewing gum comprising:
creating a base for the dissolving chewing gum by heating, cooking, and mixing a liquid, a chew/texture component, and a texture maintenance component;
adding additional components to the base;
cooling the base to incorporate a sensitive component; and
forming individual pieces of the dissolving chewing gum from the base.

2. The method of claim 1,
wherein the liquid comprises water, and
wherein the chew/texture component is selected from the group consisting of: a fat, a thickening agent, and a gelling agent.

3. The method of claim 1, wherein the additional components comprise sweetening/texture components.

4. The method of claim 1, wherein the sensitive component is selected from the group consisting of: a natural flavoring component and an active component.

5. The method of claim 1, further comprising:
pre-sealing each of the individual pieces of the dissolving chewing gum.

6. The method of claim 1, further comprising:
adding an engrossing syrup to each of the individual pieces of the dissolving chewing gum.

7. The method of claim 1, further comprising:
adding a finishing syrup and/or a coloring component to each of the individual pieces of the dissolving chewing gum.

8. The method of claim 1, further comprising:
adding a coating component, a polish, and/or a wax to each of the individual pieces of the dissolving chewing gum.

9. The method of claim 1, wherein the dissolving chewing gum contains no plastic, no animal products, and is plant-based.

10. A method to make a dissolving chewing gum without a cooking step, the method comprising:
creating a base for the dissolving chewing gum by mixing a chew/texture component and a texture maintenance component;
adding additional components to the base;
forming individual pieces of the dissolving chewing gum from the base;
covering the individual pieces of the dissolving chewing gum with a coating; and
adding a polish and/or a wax to the individual pieces of the dissolving chewing gum.

11. The method of claim 10, wherein the chew/texture component is selected from the group consisting of: a fat and a texture component.

12. The method of claim 10, wherein each of the additional components are selected from the group consisting of: a sweetening/texture component, another texture maintenance component, a natural flavoring component, and an active component.

13. The method of claim 12,
wherein an additional component of the additional components comprises the active component,
wherein the active component is released as a user chews the dissolving chewing gum product to provide a benefit to the user, and
wherein the benefit to the user is selected from the group consisting of: removing bad breath molecules of the user, providing energy to the user, reducing anxiety of the user, delivering vitamins to the user, delivering protein to the user, delivering health and/or wellness benefits the user, delivering plaque or biofilm reduction or removal benefits to the user, and providing medical components to the user.

14. The method of claim 10, wherein the dissolving chewing gum contains no plastic, no animal products, and is plant-based.
